(19) United States
(12) Patent Application Publication
(10) Pub. No.: US 2008/0038783 A1
(43) Pub. Date: Feb. 14, 2008

(54) Compositions and Methods Pertaining to Guanylation of PNA Oligomers

(75) Inventor: Eric G. Anderson, North Attleboro, MA (US)

Correspondence Address: APPLIED BIOSYSTEMS, 500 OLD CONNECTICUT PATH, FRAMINGHAM, MA, 01701, US

(73) Assignee: Applera Corporation, Framingham, MA (US)

(21) Appl. No.: 11/769,869

(22) Filed: Jun. 28, 2007

Related U.S. Application Data
(60) Provisional application No. 60/817,314, filed on Jun. 29, 2006.

(51) Int. Cl. C12P 19/34; C07H 21/00; C07K 1/00; C07K 5/00

(52) U.S. Cl. 435/91.2; 530/300; 530/333; 530/337; 536/25.32

(57) ABSTRACT

This invention is related to compositions and methods pertaining to PNA oligomers comprising one or more guanidinium moieties.

### PRIORITY AND RELATED APPLICATIONS

This application claims the benefit of priority U.S. Provisional Application Ser. No. 60/817,314 filed Jun. 29, 2006, which is incorporated herein by reference.

The section headings used herein are for organizational purposes only and are not to be construed as limiting the subject matter described in any way.

### FIELD

This invention is related to the field of the PNA oligomers, including methods for their manufacture.

### INTRODUCTION

Peptide nucleic acid (PNA) is a class of synthetic nucleobase comprising oligomers that can sequence specifically hybridize to nucleic acids and other polynucleobase strands. Hybridization between nucleobases of polynucleobase strands typically follows well-established rules for hydrogen bonding. For Watson-Crick base pairing, typically adenine base pairs with thymine and cytosine base pairs with guanine.

Recently interest in PNA oligomers comprising guanidinium groups is associated with so called “G-PNAs” (See FIGS. 1A and 1B for an illustration of the nucleobase containing subunits of DNA, (common N-[2-(aminoethyl)]glycine) PNA and G-PNA). G-PNAs are PNA oligomers comprising an N-[2-(aminoethyl)]arginine backbone as compared with the more typical N-[2-(aminoethyl)]glycine backbone (See: Ly et al., J. Am. Chem. Soc., 125: 6878-6879 (2003) and Ly et al., Chem. Comm., 2: 244-246 (2005)) According to recent publications, G-PNAs exhibit enhanced cell membrane permeability properties as compared with the more typical PNAs comprising a N-[2-(aminoethyl)]glycine backbone. According to the published procedures, G-PNAs are prepared using standard t-boc(Cbz) protection chemistry from N-[2-(aminoethyl)]arginine PNA monomers, wherein the guanidinium group of the arginine side chain is protected with a tosyl moiety. According to this method, the PNA oligomers are treated with scavengers associated with deprotection of the tosyl group, wherein said scavengers can react with certain fluorescent and/or quencher moieties, if present, to thereby decompose said certain fluorescent and/or quencher moieties. However, according to a recent report, the guanidinium group of the arginine side chain has also been protected with t-boc and Cbz protecting groups (See: FIG. 3 for an illustration of various protecting groups discussed herein).

### DRAWINGS

The skilled artisan will understand that the drawings, described below, are for illustration purposes only. The drawings are not intended to limit the scope of the present teachings in any way.

FIGS. 1A and 1B are illustrations of some uncommon nucleobases that can be incorporated into nucleic acids and PNA oligomers (including G-PNAs).

FIG. 2 is an illustration of nucleobase subunits of DNA, (commonly used) PNA and G-PNA.

FIG. 3 is an illustration of various amine protecting groups discussed herein with respect to various embodiments.

FIG. 4 is an illustration of various steps of a method for converting a primary or secondary amine group of a PNA oligomer to a guanidinium moiety.

FIG. 5 is an illustration of a PNA oligomer comprising a fluorophore and dacbyl quencher (i.e. a self-indicating PNA oligomer) and a self-indicating PNA oligomer comprising alternating N-[2-(aminoethyl)]glycine and N-[2-(aminoethyl)]arginine subunits.

FIG. 6 is an illustration of the steps of a method for forming and guanylation of a side chain amine group of a PNA subunit by building a side chain group linked to the secondary amine group of an N-[2-(aminoethyl)]glycine subunit followed by guanylation.

FIG. 7 illustrates reagents and in-situ preparation of a reagent of formula I (described herein) and its reaction with aniline to form a bis-protected guanylated aniline.

### DEFINITIONS

For the purposes of interpreting this specification, the following definitions will apply and whenever appropriate, terms used in the singular will also include the plural and vice versa. In the event that any definition set forth below conflicts with the usage of that word in any other document, including, if appropriate, any document incorporated herein by reference, the definition set forth below shall always control for purposes of interpreting this specification and its associated claims unless a contrary meaning is clearly intended (for example in interpreting the document where the term is originally used). The use of “or” herein means “and/or” unless stated otherwise or where the use of “and/or” is clearly inappropriate. The use of “a” herein means “one or more” unless stated otherwise or where the use of “one or more” is clearly inappropriate. The use of “has” “having” “comprise,” “comprises,” “comprising” “include,” “includes,” and “including” are interchangeable “open ended” terms and are not intended to be limiting. Furthermore, where the description of one or more embodiments uses the term “comprising,” those skilled in the art would understand that in some specific instances, the embodiment or embodiments can be alternatively described using language “consisting essentially or” and/or “consisting of.”

a. As used herein, “target sequence” refers to a nucleobase sequence of a polynucleobase strand sought to be determined.

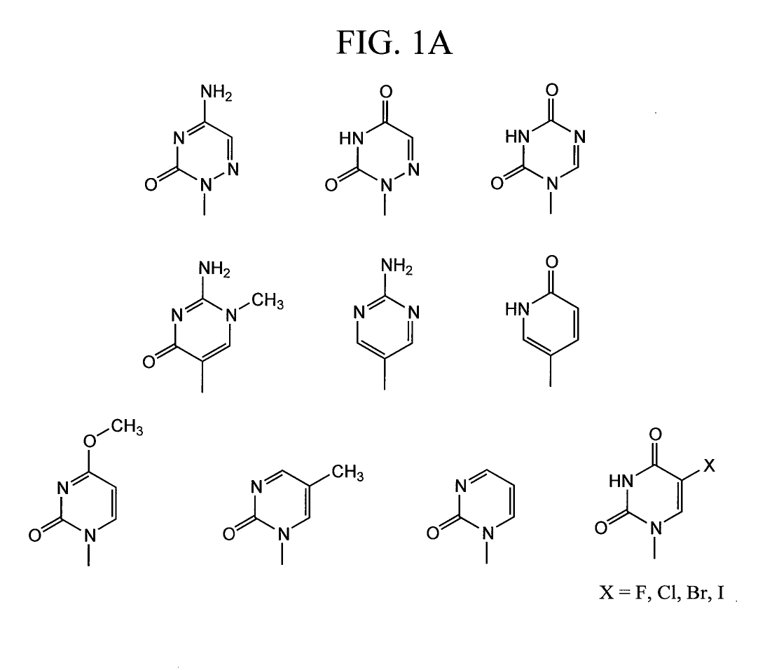

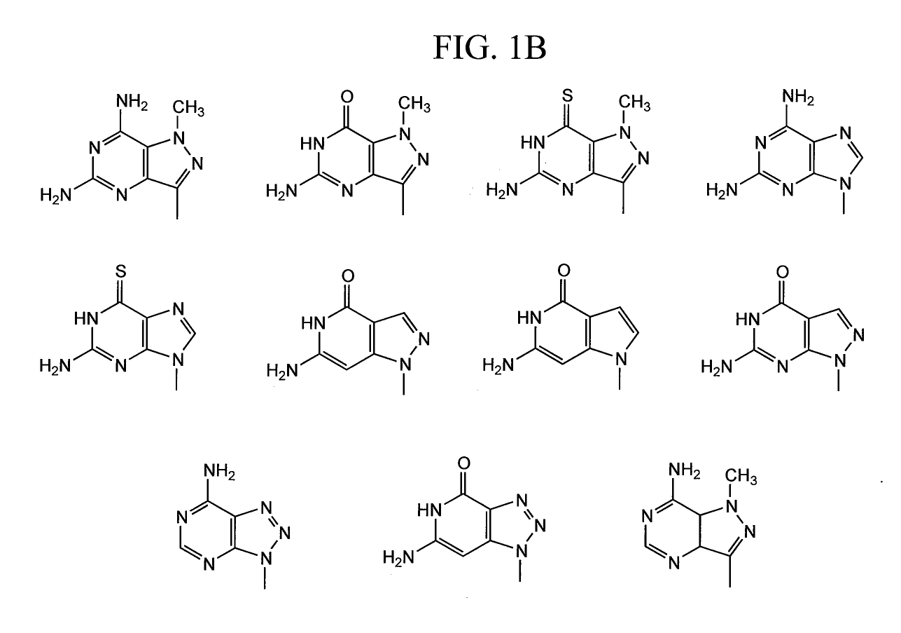

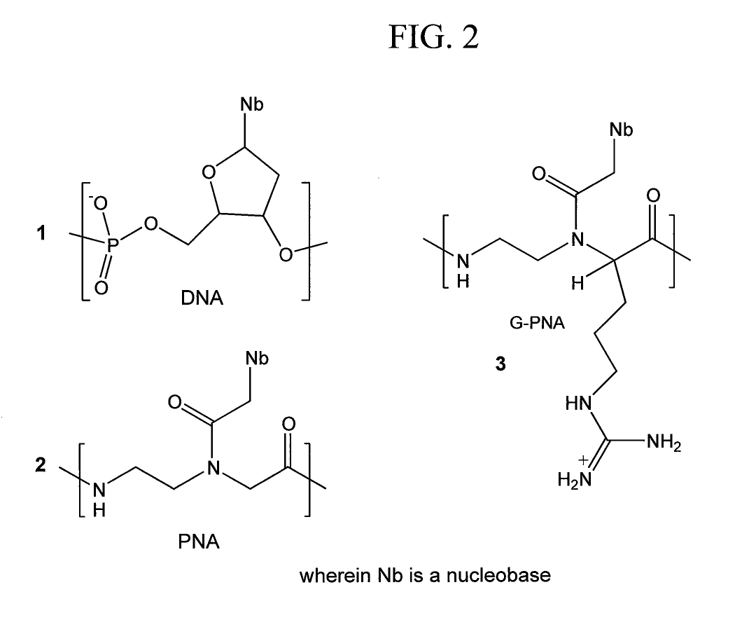

b. As used herein, “nucleobase” (abbreviated herein as “Nb”) refers to those naturally occurring and those non-naturally occurring heterocyclic moieties commonly known to those who utilize nucleic acid technology or utilize peptide nucleic acid technology to thereby generate polynucleobase strands that can sequence specifically bind to nucleic acids and other polynucleobase strands. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil, 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(8-aza-7-deazaadenine). Other non-limiting examples of suitable uncommon nucleobases include those nucleobases illustrated in FIGS. 1A and 1B (also see FIGS. 2A and 2B of U.S. Pat. No. 6,357,163 for suitable common and uncommon nucleobases).

c. As used herein, “nucleobase sequence” refers to any segment, or aggregate of two or more segments (i.e. linked polymer), of a polynucleobase strand. Non-limiting examples of suitable polynucleobase strands include oligodeoxynucleotides (e.g. DNA), oligoribonucleotides (e.g. RNA), peptide nucleic acids (PNA), PNA chimeras, nucleic acid analogs and/or nucleic acid mimics.

d. As used herein, the phrase “nucleobase containing subunit” refers to a subunit of a polynucleobase strand that comprises a nucleobase. For oligonucleotides, the nucleobase containing subunit is a nucleotide (FIG. 2 contains an illustration of an exemplary nucleic acid subunit, PNA subunit and G-PNA subunit). With reference to oligonucleotides, those of skill in the art will appreciate the form of a subunit associated with other species of polynucleobase strands (e.g. RNA, LNA, L-DNA, etc).

e. As used herein, “polynucleobase strand” refers to a complete single polymer strand comprising nucleobase-containing subunits.

f. As used herein, “nucleic acid” refers to a polynucleobase strand having a backbone formed from nucleotides, or analogs thereof. Preferred nucleic acids are DNA, RNA, L-DNA and locked nucleic acids (LNA). For the avoidance of any doubt, PNA is a nucleic acid mimic and not a nucleic acid or nucleic acid analog. PNA is not a nucleic acid since it is not formed from nucleotides.

g. As used herein, “peptide nucleic acid” or “PNA” refers to any polynucleobase strand or segment of a polynucleobase strand comprising two or more PNA subunits, including, but not limited to, any polynucleobase strand or segment of a polynucleobase strand referred to or claimed as a peptide nucleic acid in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 and 6,357,163. For the avoidance of any doubt, as used herein a PNA oligomer includes PNA chimeras and G-PNAs. For the avoidance of doubt, PNA oligomers include polymers that comprise one or more amino acid side chains linked to the backbone.

The term “peptide nucleic acid” or “PNA” shall also apply to any polynucleobase. strand or segment of a polynucleobase strand comprising two or more subunits of those nucleic acid mimics described in the following publications: Lagriffoul et al., Bioorganic & Medicinal Chemistry Letters, 4:1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000.

In some embodiments, a “peptide nucleic acid” or “PNA” is a polynucleobase strand or segment of a polynucleobase strand comprising two or more covalently linked subunits of the formula: 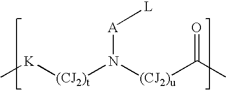
wherein, each J is the same or different and is selected from the group consisting of: H, R′, OR′, SR′, NHR′, NR′₂, F, Cl, Br and I. Each K is the same or different and is selected from the group consisting of: O, S, NH and NR′. Each R′ is the same or different and can be an alkyl group, alkenyl group, alkynyl group, heteroalkyl group, heteroalkenyl group, heteroalkynyl group or heterocycloalkyl group. For example, R′ can be methyl, ethyl, n-propyl, isopropyl, n-butyl, sec-butyl, tert-butyl, iso-butyl, n-pentyl, n-hexyl, methoxy, ethoxy, benzyl or phenyl.

Each A is a single bond, a group of the formula; —(CJ₂)ₛ— or a group of the formula; —(CJ₂)ₛC(O)—, wherein, J is defined above and each s is a integer from one to five. Each t is 1 or 2 and each u is 1 or 2. Each L is the same or different and is independently adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine), N8-(8-aza-7-deazaadenine), other naturally occurring nucleobase analogs or other non-naturally occurring nucleobases (e.g. FIGS. 1A and 1B).

In some embodiments, a PNA subunit can be a naturally occurring or non-naturally occurring nucleobase attached to the N-α-glycyl nitrogen of the N-[2-(aminoethyl)]glycine backbone through a methylene carbonyl linkage; this currently being the most commonly used form of a peptide nucleic acid subunit (e.g. FIG. 2, compound 2).

h. As used herein, “PNA chimera” means an oligomer or polymer segment comprising two or more PNA subunits and one or more nucleic acid subunits (i.e. DNA or RNA), or analogs thereof. PNA subunits and the nucleic acid subunits can be linked to the other by a covalent bond or by a linker. For example, a PNA chimera can comprise at least two PNA subunits covalently linked, via a chemical bond, to at least one 2′-deoxyribonucleic acid subunit (For exemplary methods and compositions related to PNA chimera preparation see: U.S. Pat. No. 6,063,569).

i. As used herein, “sequence specifically” refers to hybridization by base pairing through hydrogen bonding. Non-limiting examples of standard base pairing include adenine base pairing with thymine or uracil and guanine base pairing with cytosine. Other non-limiting examples of base-pairing motifs include, but are not limited to: adenine base pairing with any of: 5-propynyl-uracil, 2-thio-5-propynyl-uracil, 2-thiouracil or 2-thiothymine; guanine base pairing with any of: 5-methylcytosine or pseudoisocytosine; cytosine base pairing with any of: hypoxanthine, N9-(7-deaza-guanine) or N9-(7-deaza-8-aza-guanine); thymine or uracil base pairing with any of: 2-aminopurine, N9-(2-amino-6-chloropurine) or N9-(2,6-diaminopurine); and N8-(8-aza-7-deazaadenine), being a universal base, base pairing with any other nucleobase, such as for example any of: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine or N9-(7-deaza-guanine).

j. As used herein, the term “alkyl” refers to a straight chained or branched C₁-C₂₀ hydrocarbon or a cyclic C₃-C₂₀ hydrocarbon (i.e. a cycloalkyl group) that is completely saturated. When used herein, the term “alkyl” refers to a group that may be substituted or unsubstituted. When used herein, “alkyl” also refers to an alkyl group wherein one or more of the carbon atoms of a substituted or unsubstituted methylene group may be replaced by a silicon atom (Si). In some embodiments, alkyl groups can be a straight chained or branched C₁-C₆ hydrocarbons or cyclic C₃-C₆ hydrocarbons that are completely saturated.

k. As used herein, the term “alkylene” refers to a straight or branched alkyl chain or a cyclic alkyl group that has at least two points of attachment to at least two moieties (e.g., —{CH₂}— (methylene), —{CH₂CH₂}—, (ethylene), 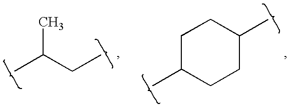
etc., wherein the brackets indicate the points of attachment). When used herein the term “alkylene” refers to a group that may be substituted or unsubstituted. In some embodiments, an alkylene group can be a C₁-C₆ hydrocarbon.

l. As used herein, the term “alkenyl” refers to straight chained or branched C₂-C₂₀ hydrocarbons or cyclic C₃-C₂₀ hydrocarbons that have one or more double bonds. When used herein, the term “alkenyl” refers to a group that can be substituted or unsubstituted. For the purposes of this specification, “alkenyl” can also refer to an alkenyl group wherein one or more of the carbon atoms of a substituted or unsubstituted methylene group have been replaced by a silicon atom (Si). In some embodiments, alkenyl groups can be straight chained or branched C₂-C₆ hydrocarbons or cyclic C₃-C₆ hydrocarbons that have one or more double bonds.

m. As used herein, the term “alkynyl” refers to straight chained or branched C₂-C₂₀ hydrocarbons or cyclic C₃-C₂₀ hydrocarbons that have one or more triple bonds. When used herein, the term “alkynyl” refers to a group that can be substituted or unsubstituted. For the purposes of this specification, “alkynyl” can also refer to an alkynyl group wherein one or more of the carbon atoms of a substituted or unsubstituted methylene group have been replaced by a silicon atom (Si). In some embodiments, alkynyl groups can be straight chained or branched C₂-C₆ hydrocarbons or cyclic C₃-C₆ hydrocarbons that have one or more triple bonds.

n. As used herein, the term “heteroalkyl” refers to an alkyl group in which one or more methylene groups in the alkyl chain is replaced by a heteroatom, or heteroatom containing group, such as —O—, —S—, —SO₂— or —NR″—, wherein R″ can be hydrogen, alkyl, alkenyl, alkynyl, aryl or arylalkyl. When used herein, the term “heteroalkyl” refers to a group that can be substituted or unsubstituted.

o. As used herein, the term “heteroalkenyl” refers to an alkenyl group in which one or more methylene groups is replaced by a heteroatom, or heteroatom containing group, such as —O—, —S—, —SO₂— or —NR″—, wherein R″ is previously defined. When used herein, the term “heteroalkenyl” refers to a group that can be substituted or unsubstituted.

p. As used herein, the term “heteroalkynyl” refers to an alkynyl group in which one or more methylene groups is replaced by a heteroatom or heteroatom containing group such as —O—, —S—, —SO₂— or —NR″—, wherein R″ is previously defined. When used herein, the term “heteroalkenyl” refers to a group that can be substituted or unsubstituted.

q. As used herein, the term “heterocycloalkyl” refers to a non-aromatic ring that comprises one or more oxygen, nitrogen and/or sulfur atoms (e.g., morpholine, piperidine, piperazine, pyrrolidine or thiomorpholine). As used herein, the term “heterocycloalkyl” refers to a group that may be substituted or unsubstituted.

r. As used herein, the term “aryl”, either alone or as part of another moiety (e.g., arylalkyl, etc.), refers to carbocyclic aromatic groups such as phenyl. Aryl groups also include fused polycyclic aromatic ring systems in which a carbocyclic aromatic ring is fused to another carbocyclic aromatic ring (e.g., 1-naphthyl, 2-naphthyl, 1-anthracyl, 2-anthracyl, etc.) or in which a carbocylic aromatic ring is fused to one or more carbocyclic non-aromatic rings (e.g., tetrahydronaphthylene, indan, etc.). As used herein, the term “aryl” refers to a group that may be substituted or unsubstituted.

s. As used herein, the term “heteroaryl” refers to an aromatic heterocycle that comprises 1, 2, 3 or 4 heteroatoms independently selected from nitrogen, sulfur and oxygen. As used herein, the term “heteroaryl” refers to a group that may be substituted or unsubstituted. A heteroaryl may be fused to one or two rings, such as a cycloalkyl, a heterocycloalkyl, an aryl, or a heteroaryl. The point of attachment of a heteroaryl to a molecule may be on the heteroaryl, cycloalkyl, heterocycloalkyl or aryl ring, and the heteroaryl group may be attached through carbon or a heteroatom. Heteroaryl groups may be substituted or unsubstituted. Examples of heteroaryl groups include imidazolyl, furyl, pyrrolyl, thienyl, oxazolyl, thiazolyl, isoxazolyl, isothiazolyl, thiadiazolyl, oxadiazolyl, pyridinyl, pyrimidyl, pyrazinyl, pyridazinyl, quinolyl, isoquinolinyl, indazolyl, benzoxazolyl, benzisooxazolyl, benzofuryl, benzothiazolyl, indolizinyl, imidazopyridinyl, pyrazolyl, triazolyl, isothiazolyl, oxazolyl, tetrazolyl, benzimidazolyl, benzothiazolyl, benzoisothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidyl, pyrazolo[3,4]pyrimidyl or benzo(b)thienyl, each of which can be optionally substituted.

t. As used herein, the term “arylalkyl” refers to an aryl group that is attached to another moiety via an alkylene linker. As used herein, the term “arylalkyl” refers to a group that may be substituted or unsubstituted.

u. As used herein, the term “heteroarylalkyl” refers to a heteroaryl group that is attached to another moiety (e.g. an alkyl or heteroalkyl group) via an alkylene linker. As used herein, the term “heteroarylalkyl” refers to a group that may be substituted or unsubstituted.

Suitable substituents for an alkyl, an alkylene, an alkenyl, an alkynyl, a heteroalkyl, a heteroalkenyl, a heteroalkynyl, an aryl, a heteroaryl, an arylalkyl, or a heteroarylalkyl group includes any substituent that is stable under the reaction conditions used in embodiments of this invention. Non limiting examples of suitable substituents include: an alkyl (e.g., methyl, ethyl, n-propyl, isopropyl, n-butyl, isobutyl, sec butyl, t-butyl, cyclohexyl etc.) group, a haloalkyl (e.g., trifluoromethyl, 2,2,2-trifluoroethyl-) group, an alkoxy (e.g., methoxy, ethoxy, etc.) group, an aryl (e.g., phenyl) group, an arylalkyl (e.g., benzyl) group, a nitro group, a cyano group, a quaternized nitrogen atom, or a halogen (e.g., fluorine, chlorine, bromine and iodine) group.

In addition, any saturated portion of an alkyl, alkenyl, alkynyl, heteroalkyl, heteroalkenyl, heteroalkynyl, arylalkyl or heteroarylalkyl group, may also be substituted with ═O, ═S, ═N—R″, wherein R″ is previously defined.

When a heteroalkyl, heteroalkenyl, heteroalkynyl, or heteroarylalkyl group contains a nitrogen atom, it may be substituted or unsubstituted. When a nitrogen atom in the aromatic ring of a heteroaryl group has a substituent, the nitrogen may be a quaternary nitrogen.

v. As used herein, “amino acid” refers to a group represented by R″′—NH—CH(R″″)—C(O)—R″′, wherein each R′″ is independently hydrogen, an aliphatic group, a substituted aliphatic group, an aromatic group, another amino acid, a peptide or a substituted aromatic group. Examples of amino acids include, but are not limited to, alanine, valine, leucine, isoleucine, aspartic acid, glutamic acid, serine, threonine, glutamine, asparagine, arginine, lysine, ornithine, proline, hydroxyproline, phenylalanine, tyrosine, tryptophan, cysteine, methionine and histidine. In some embodiments, R″″ can be hydrogen or a side-chain of a naturally-occurring amino acid. Examples of naturally occurring amino acid side-chains include methyl (alanine), isopropyl (valine), sec-butyl (isoleucine), —CH₂CH(—CH₃)₂ (leucine), benzyl (phenylalanine), p-hydroxybenzyl (tyrosine), —CH₂—OH (serine), —CHOHCH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NH₂ (ornithine), —{(CH)₂}₄NHC(═NH)NH₂ (arginine) and —CH₂-3-imidazoyl (histidine).

Side-chains of amino acids comprising a heteroatom-containing functional group, e.g., an alcohol (serine, tyrosine, hydroxyproline and threonine), an amine (lysine, ornithine, histidine and arginine), may require a protecting group to facilitate reactions discussed herein. When the heteroatom-containing functional group is modified to include a protecting group, the side-chain is referred to as the “protected side-chain” of an amino acid. Protecting groups are commonly used in peptide synthesis and these are known to, and often used by, the ordinary practitioner. For example, many suitable protecting groups, and methods for the preparation of protected amino acids, can be found in Green et al., Protecting Groups In Organic Synthesis, Third Edition, John Wiley & Sons, Inc. New York, 1999.

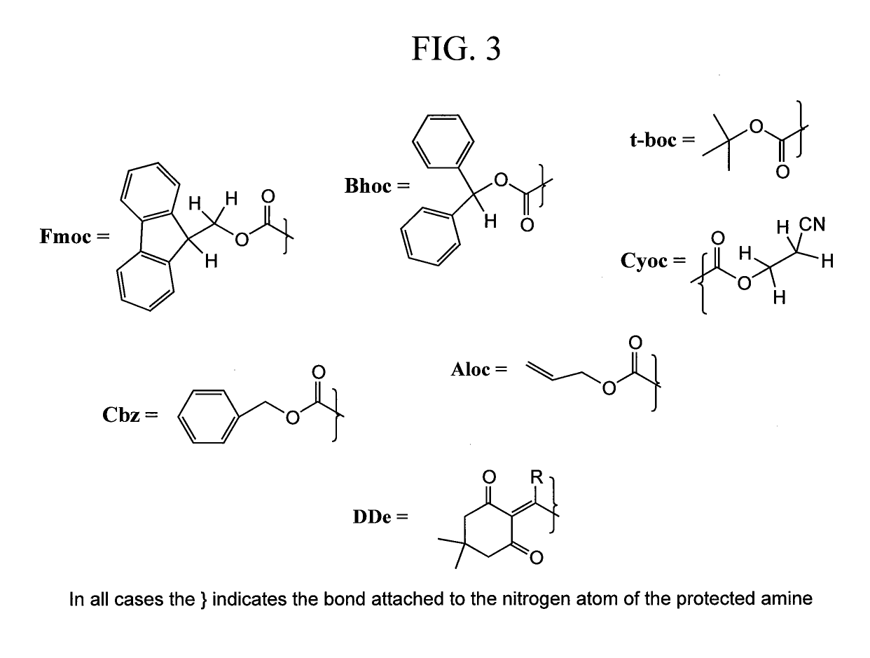

w. As used herein, the term “protecting group” refers to a chemical group that is reacted with, and bound to, a functional group in a molecule to prevent the functional group from participating in subsequent reactions of the molecule but which group can subsequently be removed to thereby regenerate the unprotected functional group. Additional reference is made to: Oxford Dictionary of Biochemistry and Molecular Biology, Oxford University Press, Oxford, 1997 as evidence that “protecting group” is a term well-established in field of organic chemistry. Some common amine protecting groups include Aloc, Bhoc, Cbz, Cyoc, DDe, Fmoc and t-boc; whose structures can be found in FIG. 3.

x. As used herein, the term “protonated form” refers to a group comprising a basic nitrogen atom that, under conditions based upon the pK of the basic nitrogen, reacts with a proton from bulk fluid to thereby reversibly produce a positively charged group comprising an additional hydrogen atom.

y. As used herein, the phase “under basic conditions” refers to conditions under which the primary or secondary amine group is substantially unprotonated and therefore available for reaction as a nucleophile. Basic conditions can be produced by adding non-nucleophilic organic bases such as triethylamine or N,N′-diisopropylethylamine. Inorganic bases such as sodium, potassium or cesium carbonate can also be used to produce suitable basic conditions under which a primary or secondary amine is substantially unprotonated. By ‘substantially unprotonated’ we mean that at least 80% of the compound in a representative sample exists in the unprotonated form.

z. As used herein, the term “leaving group” refers to any atom or group, charged or uncharged, that departs during a substitution or displacement reaction from what is regarded as the residual or main part of the substrate of the reaction. Additional reference is made to: Oxford Dictionary of Biochemistry and Molecular Biology, Oxford University Press, Oxford, 1997 as evidence that “leaving group” is a term well-established in field of organic chemistry.

aa. As used herein, “support bound” refers to a PNA oligomer immobilized on or to a solid support.

ab. As used herein “support”, “solid support” or “solid carer” refers to any solid phase material upon which a PNA oligomer is synthesized, attached, ligated or otherwise immobilized. Support encompasses terms such as “resin”, “solid phase”, “surface” and “solid support”. A support may be composed of organic polymers such as polystyrene, polyethylene, polypropylene, polyfluoroethylene, polyethyleneoxy, and polyacrylamide, as well as co-polymers and grafts thereof. A support may also be inorganic, such as glass, silica, controlled-pore-glass (CPG), or reverse-phase silica. The configuration of a support may be in the form of beads, spheres, particles, granules, a gel, or a surface. Surfaces may be planar, substantially planar, or non-planar. Supports may be porous or non-porous, and may have swelling or non-swelling characteristics. A support may be configured in the form of a well, depression or other container, vessel, feature or location. A plurality of supports may be configured in an array at various locations, addressable for robotic delivery of reagents, or by detection means including scanning by laser illumination and confocal or deflective light gathering.

### DESCRIPTION

### i.) General

It is to be understood that the discussion set forth below in this “General” section can pertain to some, or to all, of the various embodiments of the invention described herein.

PNA Synthesis:

Methods for the chemical assembly of PNAs are known (See for example: U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053 and 6,107,470). As a general reference for PNA synthesis methodology please see: Nielsen et al., Peptide Nucleic Acids; Protocols and Applications, Horizon Scientific Press, Norfolk England (1999).

Chemicals and instrumentation for the support bound automated chemical assembly of peptide nucleic acids are available. Both labeled and unlabeled PNA oligomers are likewise available from commercial vendors of custom PNA oligomers. Chemical assembly of a PNA is analogous to solid phase peptide synthesis, wherein at each cycle of assembly the oligomer possesses a reactive alkylamino terminus that can be condensed with the next synthon to be added to the growing polymer. Because standard peptide chemistry is utilized, natural and non-natural amino acids can be routinely incorporated into a PNA oligomer. Because a PNA is a polyamide, it has a C-terminus (carboxyl terminus) and an N-terminus (amino terminus). For the purposes of the design of a hybridization probe suitable for antiparallel binding to the target sequence (the preferred orientation), the N-terminus of the probing nucleobase sequence of the PNA probe is the equivalent of the 5′-hydroxyl terminus of an equivalent DNA or RNA oligonucleotide. The orientation of hybridization is not a limitation however, since PNA oligomers are also known to bind in parallel orientation to both nucleic acids and other PNA oligomers.

PNA Labeling:

Non-limiting methods for labeling PNA oligomers are described in U.S. Pat. Nos. 6,110,676, 6,355,421, 6,361,942 and 6,485,901 or are otherwise known in the art of PNA synthesis. Other non-limiting examples for labeling PNA oligomers are also discussed in Nielsen et al., Peptide Nucleic Acids; Protocols and Applications, Horizon Scientific Press, Norfolk England (1999). PNA oligomers and oligonucleotides can also be labeled with proteins (e.g. enzymes) and peptides as described in U.S. Pat. No. 6,197,513. Thus, a variety of labeled PNA oligomers can be prepared or purchased from commercial vendors.

Labels:

PNA oligomers can comprise a label. Non-limiting examples of detectable moieties (labels) that can be used to label polynucleobase strands (e.g. PNA oligomers) include a dextran conjugate, a branched nucleic acid detection system, a chromophore, a fluorophore, a spin label, a radioisotope, an enzyme, a hapten, an acridinium ester or a chemiluminescent compound. Other suitable labeling reagents and preferred methods of attachment would be recognized by those of ordinary skill in the art of PNA, peptide or nucleic acid synthesis.

Non-limiting examples of haptens include 5(6)-carboxyfluorescein, 2,4-dinitrophenyl, digoxigenin, and biotin.

Non-limiting examples of fluorochromes (fluorophores) include 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.) or the Alexa dye series (Molecular Probes, Eugene, Oreg.).

Non-limiting examples of enzymes include polymerases (e.g. Taq polymerase, Klenow DNA polymerase, T7 DNA polymerase, Sequenase, DNA polymerase 1 and phi29 polymerase), alkaline phosphatase (AP), horseradish peroxidase (HRP), soy bean peroxidase (SBP)), ribonuclease and protease.

Spacer/Linker Moieties:

PNA oligomers can comprise a spacer and/or linker moiety. Generally, spacers are used to minimize the adverse effects that bulky labeling reagents might have on hybridization properties of probes. Linkers typically induce flexibility and randomness into the polynucleobase strand or otherwise link two or more nucleobase sequences of a polynucleobase strand. Preferred spacer/linker moieties for the polynucleobase strands described herein can comprise one or more aminoalkyl carboxylic acids (e.g. aminocaproic acid), the side chain of an amino acid (e.g. the side chain of lysine or ornithine), natural amino acids (e.g. glycine), aminooxyalkylacids (e.g. 8-amino-3,6-dioxaoctanoic acid), alkyl diacids (e.g. succinic acid), alkyloxy diacids (e.g. diglycolic acid) or alkyldiamines (e.g. 1,8-diamino-3,6-dioxaoctane). Spacer/linker moieties can also incidentally or intentionally be constructed to improve the water solubility of the polynucleobase strand (For example see: Gildea et al., Tett. Lett. 39: 7255-7258 (1998) and U.S. Pat. Nos. 6,326,479 and 6,770,442).

Deprotection of Tosyl Amines

The Tosyl group (p-tolylsulfonyl) can be used to protect the guanidino moiety of arginine during t-boc peptide/PNA synthesis. Its removal can be performed concomitantly during final cleavage of the peptide/PNA, when hydrogen fluoride (HF)-anisole is used. Deprotection of the Tosyl group using a trifluoromethanesulfonic acid (TFMSA)-thioanisole based cleavage cocktail has also been achieved (See: Kiso et al., J.C.S. Chem. Comm. 770: 1063-1064 (1980)). An advantage of the latter cleavage procedure is that no special apparatus for handling HF is required. The rate of Tosyl deprotection is dependent upon the nucleophilicity of the scavenger employed: thioanisole>anisole>diemthyl sulfide>phenol-o-cresol-ethanedithiol (See: Kiso, et al.). However, the scavengers can themselves react with various compounds, including some fluorescent dyes and quenchers.

The inventor has determined that a cleavage mixture comprising 6:2:1.5:0.5 trifluoroacetic acid (TFA):TFMSA:thioanisole:H₂O can be used to deprotect the Tosyl group from arginine-containing peptides and PNAs.

Energy Transfer

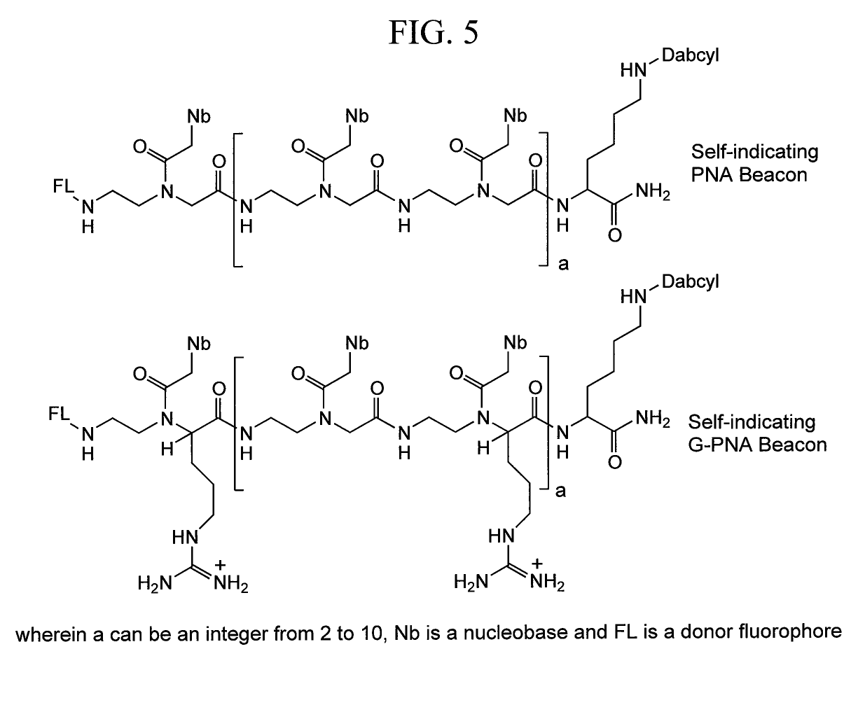

Energy transfer can be used in hybridization analysis. For energy transfer to be useful in determining hybridization, there should be an energy transfer set comprising at least one energy transfer donor and at least one energy transfer acceptor moiety. For example, a self-indicating PNA oligomer can be labeled with a donor moiety (typically a donor fluorophore) and acceptor moiety (typically a quencher acceptor) in a manner that is described in U.S. Pat. No. 6,326,479, 6,355,421 or 6,485,901 (also see FIG. 5). Often, the energy transfer set will include a single donor moiety and a single acceptor moiety, but this is not a limitation. An energy transfer set may contain more than one donor moiety and/or more than one acceptor moiety. The donor and acceptor moieties operate such that one or more acceptor moieties accepts energy transferred from the one or more donor moieties or otherwise quenches the signal from the donor moiety or moieties. Thus, in some embodiments, both the donor moiety(ies) and acceptor moiety(ies) are fluorophores. Though the previously listed fluorophores (with suitable spectral properties) might also operate as energy transfer acceptors the acceptor moiety can also be a quencher moiety such as 4-((−4-(dimethylamino)phenyl)azo) benzoic acid (dabcyl). The labels of the energy transfer set can be linked at the oligomer block termini or linked at a site within the PNA oligomer. In one embodiment, each of two labels of an energy transfer set can be linked at the distal-most termini of the PNA oligomer. PNA oligomers comprising donor and acceptor moieties can also comprise at least one linked amino acid that comprises a charged group at physiological pH.

Transfer of energy between donor and acceptor moieties may occur through any energy transfer process, such as through the collision of the closely associated moieties of an energy transfer set(s) or through a non-radiative process such as fluorescence resonance energy transfer (FRET). For FRET to occur, transfer of energy between donor and acceptor moieties of a energy transfer set requires that the moieties be close in space and that the emission spectrum of a donor(s) have substantial overlap with the absorption spectrum of the acceptor(s) (See: Yaron et al. Analytical Biochemistry, 95: 228-235 (1979) and particularly page 232, col. 1 through page 234, col. 1). Alternatively, collision mediated (radiationless) energy transfer may occur between very closely associated donor and acceptor moieties whether or not the emission spectrum of a donor moiety(ies) has a substantial overlap with the absorption spectrum of the acceptor moiety(ies) (See: Yaron et al., Analytical Biochemistry, 95: 228-235 (1979) and particularly page 229, col. 1 through page 232, col. 1). This process is referred to as intramolecular collision since it is believed that quenching is caused by the direct contact of the donor and acceptor moieties (See: Yaron et al.). It is to be understood that any reference to energy transfer in the instant application encompasses all of these mechanistically-distinct phenomena. It is also to be understood that energy transfer can occur though more than one energy transfer process simultaneously and that the change in detectable signal can be a measure of the activity of two or more energy transfer processes. It is to be understood that energy transfer can also occur by mechanisms that have not been described. Accordingly, the mechanism of energy transfer is not a limitation of this invention.

Detecting Energy Transfer in a Self-Indicating PNA Oligomer:

In some embodiments, the PNA oligomers are self-indicating. For example, a self-indicating PNA oligomer can be labeled in a manner that is described in U.S. Pat. No. 6,326,479, 6,355,421 or 6,485,901. The PNA oligomers depicted in FIG. 5 are examples of a self-indicating PNA oligomers.

Hybrid formation between a self-indicating PNA oligomer and a polynucleobase strand can be monitored by measuring at least one physical property of at least one member of the energy transfer set that is detectably different when the PNA oligomer/nucleic acid (NA) complex is formed as compared with when the PNA oligomer exists in a non-hybridized state. We refer to this phenomenon as the self-indicating property of the PNA oligomer. This change in detectable signal results from the change in efficiency of energy transfer between donor and acceptor moieties caused by hybridization of the PNA oligomer to the nucleic acid sequence.

For example, the means of detection can involve measuring fluorescence of a donor or acceptor fluorophore of an energy transfer set. For example, the energy transfer set may comprise at least one donor fluorophore and at least one acceptor (fluorescent or non-fluorescent) quencher such that the measure of fluorescence of the donor fluorophore can be used to detect, identify and/or quantify hybridization of the PNA oligomer to the nucleic acid.

In some embodiments, the energy transfer set comprises at least one donor fluorophore and at least one acceptor fluorophore such that the measure of fluorescence of either, or both, of at least one donor moiety or one acceptor moiety can be used to detect, identify and/or quantify hybridization of the PNA oligomer to the nucleic acid.

Detectable and Independently Detectable Moieties/Multiplex Analysis:

In some embodiments, a multiplex hybridization assay can be performed. In a multiplex assay, numerous conditions of interest are simultaneously or sequentially examined. Multiplex analysis relies on the ability to sort sample components or the data associated therewith, during or after the assay is completed. In performing a multiplex assay, one or more distinct independently detectable moieties can be used to label two or more different PNA oligomers that are used in an assay. By independently detectable we mean that it is possible to determine one detectable moiety independently of, and in the presence of, the other detectable moiety. The ability to differentiate between and/or quantify each of the independently detectable moieties provides the means to multiplex a hybridization assay because the data correlates with the hybridization of each of the distinct, independently labeled PNA oligomer to a particular target sequence sought to be determined in the sample. Consequently, the multiplex assays can, for example, be used to simultaneously and/or sequentially detect the presence, absence, number, position and/or identity of two or more target sequences in the same sample and in the same assay. For example, the condition or conditions of interest could be determined in a multiplex PCR assay wherein the PNA oligomers are probes and wherein each probe can determine whether or not a target sequence of interest exists in the sample that is analyzed.

### ii) Various Embodiments of the Invention

a. Methods:

i) Guanylation

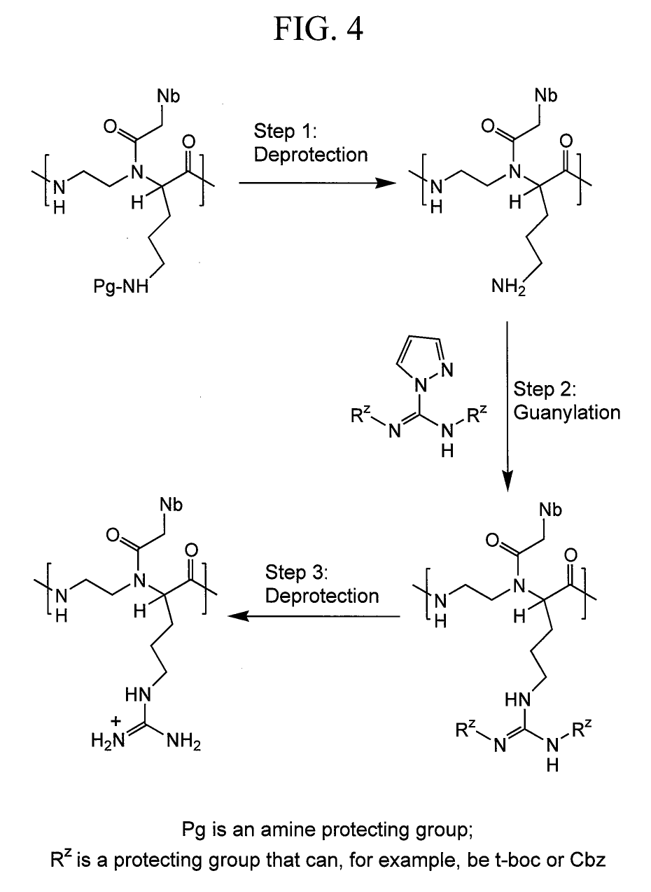

In some embodiments, this invention pertains to a general method for guanylating one or more primary or secondary amine groups of a PNA oligomer. Guanylation of amine groups can improve various properties of the PNA oligomers, such as solubility and/or cell membrane permeability. PNA oligomers possessing good solubility and/or membrane permeability characteristics can be used as antisense agents and/or used as probes in amplification reactions such as in the polymerase chain reaction (PCR). The process for guanylating amine groups of PNA oligomers disclosed herein is straightforward and can be performed on PNA oligomers in solution as well as PNA oligomers that are support bound. One or more steps that might be part of the conversion of a primary or secondary amine group of a PNA oligomer to a guanydinyl group are illustrated in FIG. 4.

Thus, in some embodiments, this invention pertains to a method comprising reacting one or more primary or secondary amine groups of a PNA oligomer comprising a backbone with a reagent of formula I: 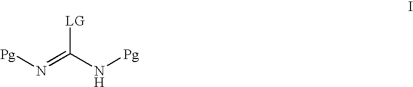
under basic conditions to thereby form a protected guanidinium group from each of said one or more reacted amine groups (See: FIG. 4, Step 2). According to the method, each Pg is independently the same or a different amine protecting group and LG is a leaving group that can be displaced by reaction with the one or more amine groups of the PNA oligomer. Reaction of a reagent of formula I with each amine group of the PNA oligomer can form a protected guanidinium group of formula I′: 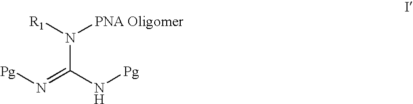
wherein R₁ can be hydrogen (if a primary amine) or an alkyl, alkenyl, alkynyl, heteroalkyl, heteroalkenyl, heteroalkynyl, heterocycloalkyl, aryl, heteroaryl, arylalkyl or heteroarylalkyl group (if a secondary amine) and each group, Pg, is previously defined.

If the amine group to be reacted with the reagent of formula I is protected from reaction, the method can, in some embodiments, further comprise deprotecting the one or more amine groups prior to their reaction with the reagent of formula I (See: FIG. 4, step 1). For the avoidance of doubt, this step is optional unless the amine group is protected from reaction towards the reagent of formula I. For example, if the amine is protected with a carbamate protecting group such as Aloc, Bhoc, Cbz, Cyoc, Fmoc or t-boc, the carbamate protecting group would need to be removed before reaction with a reagent of formula I can occur.

Whether or not the amine of the PNA oligomer requires deprotection, in some embodiments, the method further comprises deprotecting one or both of the amine protecting groups, Pg, of the guanylated PNA oligomer. As discussed, each amine protecting group, Pg, can the same or a different amine protecting group. For example, each Pg can be independently, Aloc, Bhoc, Cbz, Cyoc, DDe, Fmoc or t-boc. If the PNA oligomer is support bound, deprotection of one or both of the amine protecting groups, Pg, can, in some embodiments, be performed simultaneously with cleavage of the PNA oligomer from the solid support.

With reference to the reagent of formula I, LG can be any leaving group that can be displaced by reaction with the primary or secondary amine group of the PNA oligomer. For example, LG can be a group of formula II: 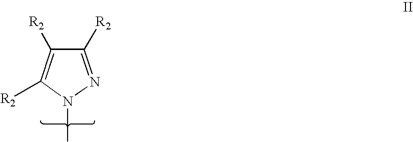
wherein the bracket “}” is used to indicate the points of attachment of the leaving group of formula II to the remainder of the reagent of formula I and each R₂ is, independently of the others, hydrogen, fluorine, chlorine, bromine or iodine or an alkyl, alkenyl, alkynyl, heteroalkyl, heteroalkenyl, heteroalkynyl, heterocycloalkyl, aryl, heteroaryl, arylalkyl, heteroarylalkyl, nitro, cyano or sulfo group. For example, the reagent of formula I or II can be N,N′-bis-(t-boc)-guanylpyrazole (V) or N,N′-bis-Cbz-guanylpyrazole (VI): 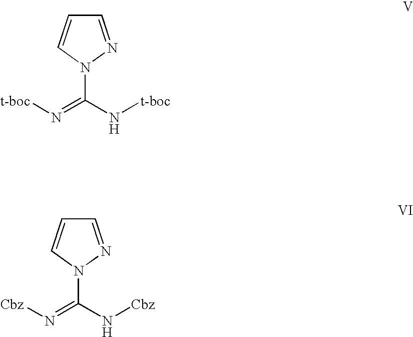
wherein t-boc and Cbz are previously defined (see: FIG. 3).

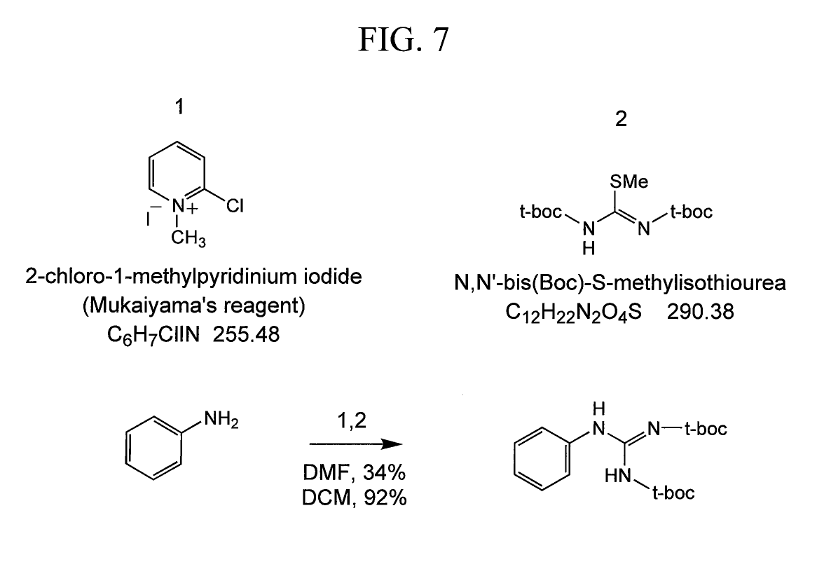

In some embodiments, the reagent of formula I can be generated in situ for reaction with the amine group of the PNA oligomer. For example, the reagent of formula I can be generated in situ by combining reagents of formulas III and IV; 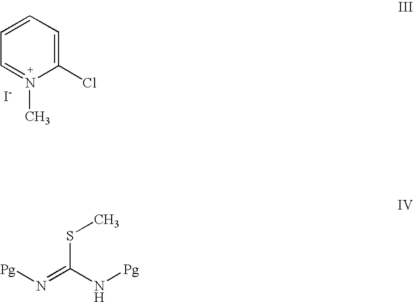
wherein each Pg is the same or a different amine protecting group (See FIG. 7 and Yong et al., J. Org. Chem. 62: 1540-1542 (1997)).

In some embodiments, the reagent of formula I can be reacted with any primary amine and secondary amine of the PNA oligomer. For example, the reacted amine group of the PNA oligomer can be a terminal amine group or a side chain amine group. In some embodiments, both the N-terminal amine group and one or more side chain amine groups can be reacted with the reagent of formula I.

In some embodiments, at least one of the amine groups can be the N-terminal amine group of the PNA oligomer. The N-terminal amine group can be a primary or a secondary amine group. Thus, in some embodiments, reaction of the reagent of formula I with the N-terminal amine group of the PNA oligomer can form a PNA oligomer represented by formula X: 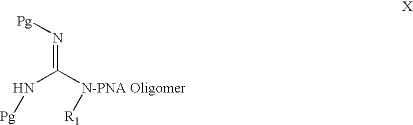
wherein R₁ and each Pg are previously defined. In some embodiments, deprotection of one or both protecting groups, Pg, of the protected guanidinium group can be performed. For example, deprotection of both protecting groups, Pg, after reaction of the PNA oligomer with the reagent of formula I according to claim 1, can produce a guanylated N-terminus of the PNA oligomer of formula X′ or a protonated version thereof represented by formula X″.

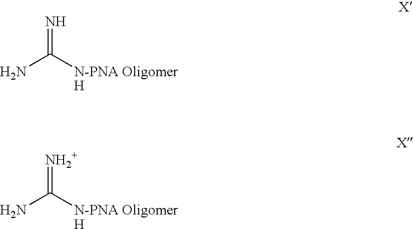

In some embodiments, at least one of the amine groups can be an amine group linked to a side chain of a subunit of the backbone of the PNA oligomer. In some embodiments, two or more of the reactive amine groups can be linked to a side chain of different subunits of the backbone of the PNA oligomer. For example, the amine group or groups can be linked to a side chain of a subunit of the backbone of the PNA oligomer wherein the backbone subunit has the formula VII: 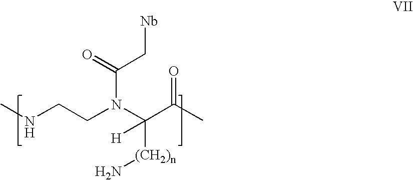
wherein n is an integer from 1 to 6 and Nb is any nucleobase. This method is particularly useful for converting PNA oligomers comprising an N-[2-(aminoethyl)]ornithine subunit to PNA oligomers comprising an N-[2-(aminoethyl)]arginine subunit. Thus, in some embodiments, this invention pertains to the practice of the above described method, wherein one amine group is linked to the side chain of a subunit of the backbone of the PNA oligomer and wherein the backbone subunit has the formula VIII: 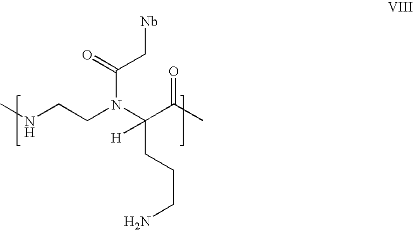
wherein Nb is any nucleobase. Reaction of the side chain amine group of the subunit of formula VIII with a reagent of formula I as described above in combination with subsequent deprotection of both protecting groups, Pg, of the protected guanidinium group, can produce a backbone subunit of formula VIII′ or a protonated version thereof represented by formula VIII″: 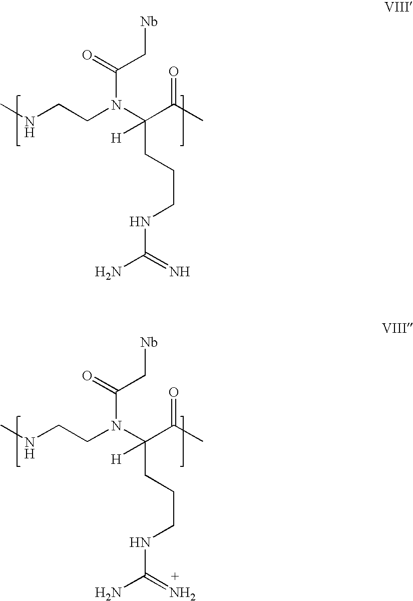
wherein Nb is any nucleobase.

In some embodiments, the amine group or groups can be linked to a side chain of a subunit of the backbone of the PNA oligomer wherein the backbone subunit has the formula IX: 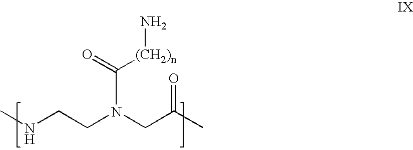
wherein n is an integer from 1 to 6. Reaction of the side chain amine group with a reagent of formula I, as described above in combination with subsequent deprotection of both protecting groups, Pg, of the protected guanidinium group, can produce a backbone subunit of formula IX′ or a protonated version thereof represented by formula IX″: 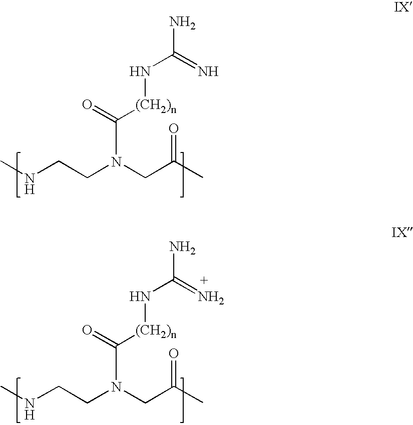
wherein n is an integer from 1 to 6.

Because the method for producing the guanylated PNA oligomers avoids the need to use harsh deprotection conditions commonly used to produce, for example, PNA oligomers comprising an N-[2-(aminoethyl)]arginine subunit, it is possible to prepare labeled PNA oligomers comprising labels (e.g. fluorophores or quenchers) that are unstable to scavengers commonly used in the deprotection of tosyl protecting groups. Accordingly, in some embodiments, this invention pertains to producing guanylated PNA oligomers comprising at least one covalently linked fluorophore and/or at least one covalently linked quencher using the aforementioned method to guanylate the PNA oligomer. Accordingly, the disclosure provided herein permits the simplified manufacture of guanylated PNA oligomers comprising labels that are unstable to current manufacturing methodologies.

Because it is not necessary for every subunit of a PNA oligomer to be guanylated in order for it to exhibit increased solubility and cell membrane permeability, in some embodiments, less than all of the subunits of the PNA oligomer will comprise a guanidinium side chain. For example, PNA oligomers can be prepared using the method described above, wherein the PNA oligomer comprises alternating subunits wherein every other PNA subunit comprises an amine group linked to a side chain of the PNA backbone and wherein each said side chain amine group is capable of being guanylated by reaction with the reagent of formula I under basic conditions. An exemplary guanylated PNA oligomer produced by practice of said aforementioned method can comprise four PNA subunits of formula XI′: 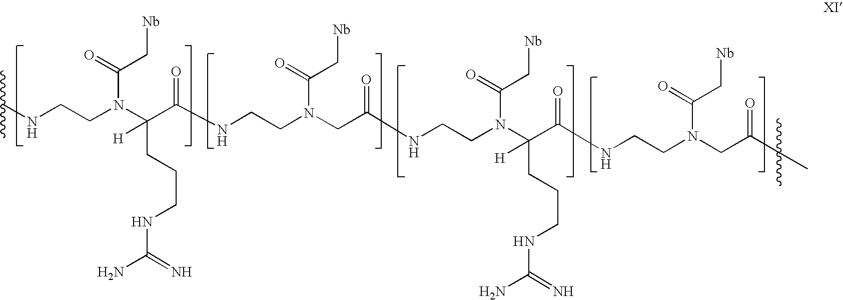
or the protonated form thereof represented by formula XI″: 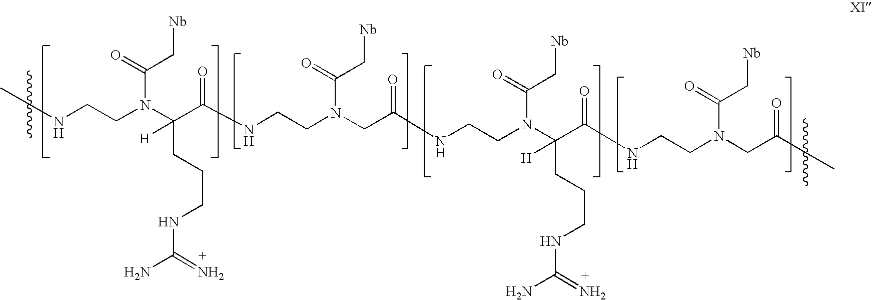
wherein the structure within each [ ] represents a PNA backbone subunit, each [symbol] indicates a point of attachment to the remainder of the PNA oligomer and each Nb is any nucleobase, wherein each nucleobase can be the same of different as compared with the other nucleobases of the PNA oligomer.

ii. Methods Involving Nucleic Acid Amplification

In some embodiments, any PNA oligomers, comprising at least one guanidinium group (protonated or unprotonated depending on pH), that are prepared according to the method previously described can potentially be used as probes in nucleic amplification reactions. This includes PNA oligomers comprising a covalently linked fluorophore and/or covalently linked quencher. Non-limiting examples of nucleic acid amplification reactions include, but are not limited to, Polymerase Chain Reaction (PCR), Ligase Chain Reaction (LCR), Strand Displacement Amplification (SDA), Transcription-Mediated Amplification (TMA), Q-beta replicase amplification (Q-beta) and Rolling Circle Amplification (RCA). Those of ordinary skill in the art may be familiar with other nucleic acid amplification methods.

Accordingly, in some embodiments, this invention pertains to a method comprising performing an amplification using a PNA oligomer probe capable of sequence specifically hybridizing to a target sequence in a nucleic acid of interest, wherein said nucleic acid of interest can be amplified, if present, and wherein the PNA oligomer probe comprises at least one guanylated amine and at least one covalently linked fluorophore and/or covalently linked quencher that is sensitive to the scavengers used during deprotection of tosyl amines. For example, the PNA oligomer can have the general formula XV, or the protonated form thereof represented by formula XV′: 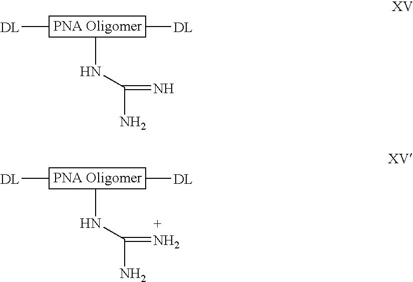
wherein each DL is independently of the other, a covalently linked fluorophore or a covalently linked quencher provided that only one of the two DLs can be a quencher.

In some embodiments, the PNA oligomer can comprise at least one PNA subunit of formula VIII′: 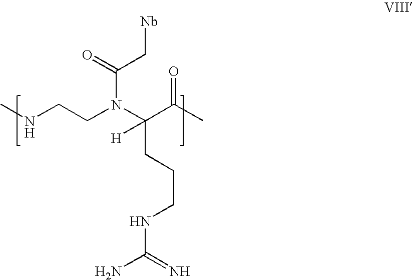
or a protonated version thereof represented by formula VIII″: 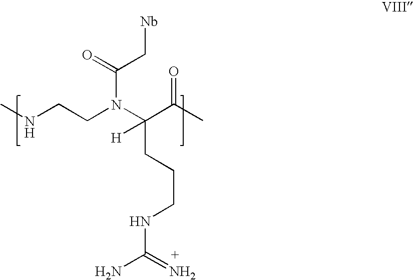
and at least one covalently linked fluorophore and/or covalently linked quencher that is sensitive to the scavengers used during deprotection of tosyl amines, wherein Nb is any nucleobase. For example, the PNA oligomer can comprise alternating subunits wherein every other PNA subunit has the formula VIII′: 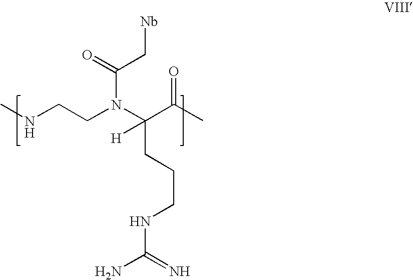
or a protonated version thereof represented by formula VIII″: 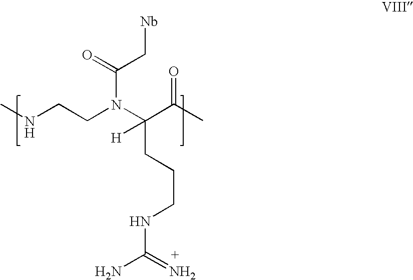
wherein Nb is any nucleobase. In some embodiments, the method can be operated in multiplex mode using two or more independently detectable PNA oligomers. In some embodiments, the nucleic acid amplification reaction is a PCR reaction. In some embodiments, the PCR reaction is analyzed in real-time. In some embodiments, the PCR reaction is analyzed at the end-point. In some embodiments, the PCR reaction is analyzed both in real-time and at the end-point.

b. Compositions

Because the method of producing guanylated PNA oligomers disclosed herein avoids the need to deprotect one or more tosylated guanidinium groups as typically performed by previously known methods, Applicants invention permits the manufacture of PNA oligomers comprising one or more guanidinium groups (protonated or unprotonated depending on pH) and one or more covalently linked fluorophores and/or quencher moieties that are unstable to the scavengers used to deprotect tosyl amine groups. For example the quencher, dabcyl, is unstable to the scavengers used to deprotect tosyl amines.

PNA oligomers comprising fluorophores and/or quencher moieties are useful as probes in various nucleic acid detection methods. For example, PNA oligomers comprising fluorophores are particularly useful in detecting organisms by in-situ hybridization (See for example, U.S. Pat. Nos. 6,649,349, 6,656,687, 6,664,045 and 7,060,432). PNA oligomers comprising fluorophore and quencher moieties are particularly useful as probes in amplification reactions such as in real-time and/or end point analysis in PCR reactions (See for example, U.S. Pat. Nos. 6,355,421, 6,361,942, 6,485,9012). Consequently, this invention pertains to any PNA oligomer comprising at least one guanidinium group and at least one covalently linked fluorophore and/or covalently linked quencher, where at least one of said fluorophore or quencher is unstable to the scavengers used to deprotect tosyl amines.

Thus, in some embodiments, this invention pertains to a PNA oligomer comprising at least one PNA subunit of formula VIII′: 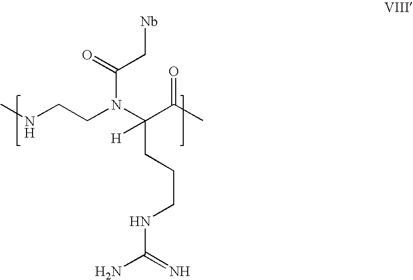
or a protonated version thereof represented by formula VIII″: 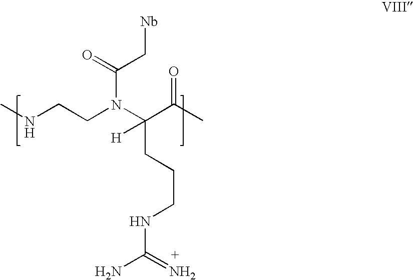
and at least one covalently linked fluorophore and/or covalently linked quencher that is sensitive to the scavengers used during deprotection of tosyl amines, wherein Nb is any nucleobase. For example, the PNA oligomer can comprise at least one covalently linked dabcyl quencher and at least one PNA subunit of formula VIII′ or a protonated version thereof represented by formula VIII″. In some embodiments, the PNA oligomer can comprise alternating subunits wherein every other PNA subunit has the formula VIII′ or a protonated version thereof represented by formula VIII″.

### 6. EXAMPLES

Aspects of the present teachings can be further understood in light of the following examples, which should not be construed as limiting the scope of the present teachings in any way.

### Example 1

### Guanylation of the N-terminal Amine of a PNA Oligomer

PNA Sequence(s):

“Test 17 PNA Sequence”: H-CGG-ACT-AAG-TCC-ATT-GC-NH₂, MW=4605.44

The Test 17 PNA Sequence was prepared using t-Boc chemistry (5 μmol) on the Applied Biosystems (ABI) 433A peptide synthesizer according to the protocol used for the production of commercial PNA oligomers. Mass spectrometry (MS) analysis of a test cleavage predominately showed full-length product along with several acetylated failure sequences.

A 12 mg portion of the resin bound Test 17 PNA Sequence, on methylbenzhydrylamine (MBHA) resin, was used for the post-synthetic guanylation study. Assuming a resin loading of 0.1 μmol/mg and 100% coupling efficiency, it was estimated that 1.2 μmol of PNA was present in the 12 mg of MBHA resin. Based upon this assumption, 18.6 mg (˜60-fold excess) of the guanylating reagent, N,N′-bis-(t-boc)-guanylpyrazole (310.35 g/mol, Fluka P/N 17262) was dissolved in 200 μL of DMF, to which 10.5 μL (30 equivalents) of N,N′-diisopropylethylamine (DIEA) was added. The mixture was added to the resin bound Test 17 PNA Sequence and allowed to react overnight with constant shaking.

Using a Millipore 0.2 um TEFLON® (PTFE) spin-cartridge, the resin was washed repeatedly with DCM. The PNA was cleaved from the resin using a trifluoroacetic acid (TFA): trifluoromethanesulfonic acid (TFMSA):m-cresol (7:2:1, 400 μL) mixture for 2 hours. The liberated PNA was then precipitated with diethyl ether, washed 3 times with diethyl ether and dried on a heat block. The crude pellet was reconstituted in 400 μL of 0.1% TFA, 25% acetonitrile (ACN)/H₂O.

MS analysis of the crude PNA was performed using matrix assisted laser desorption ionization-time of flight-mass spectrometry (MALDI-TOF-MS) using a Voyager DE-PRO (ABI) and sinapinic acid as the matrix. Guanylation of the N-terminal amine was shown as the addition of 42.05 Da relative to the unlabeled sequence (observed m/z=4645.08, calculated m/z=4647.49).

### Example 2

### Guanylation of a Side Chain Amine within a Subunit of a PNA Oligomer

Overview: This study investigated the guanylation of side chain-based amino groups contained within a subunit of a PNA oligomer. This example is intended to demonstrate that side chain amines can by guanylated, such as for the conversion of the 6-amine of an PNA N-[2-(aminoethyl)]ornithine subunit, to an N-[2-(aminoethyl)]arginine PNA subunit, wherein a model compound is used to demonstrate feasibility of this conversion.

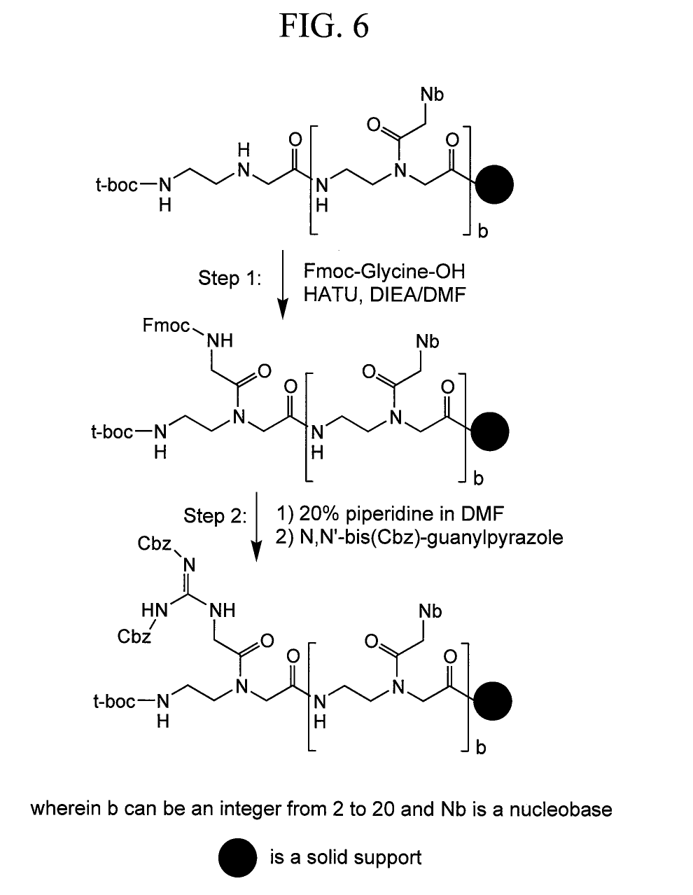

In this case, the glycine derivative Fmoc-N—(N-β-t-boc-aminoethyl)-Gly-OH (a.k.a. “t-boc-aeg(Fmoc)-OH”, Bachem) was coupled to the 2-aminoethylglycine PNA backbone within the oligomer to thereby generate an oligomer to which a compound containing a side chain amine could be coupled to thereby generate the side chain amine group suitable for guanylation. After removal of the Fmoc protecting group of the backbone secondary nitrogen atom of the linked Fmoc-N—(N-β-t-boc-aminoethyl)-Glycine moiety, the side chain amine group was introduced into the PNA oligomer by coupling of either Fmoc-glycine or Fmoc-β-alanine to the deprotected secondary amine (See: FIG. 6, Step 1). Following Fmoc removal of the primary amine of the glycine or β-alanine moiety, the primary amino group was guanylated using N,N′-bis-Cbz-guanylpyrazole (See: FIG. 6, Step 2). An overview of this process using Glycine is described in more detail below.

PNA Oligomer Sequences:

“GT-1” PNA: H-TCT-CTT-T-OOO-T-TTC-TCT-Lys-NH₂, MW=4248.22

“GT-1a” PNA: t-boc-TT-T-OOO-T-TTC-TCT-Lys-NH

“GT-2-X” PNA: H-TXT-XTT-T-OOO-T-TTC-TCT-Lys-NH₂

where X=Glycine or β-Alanine

The three “GT” PNA oligomers were prepared using t-boc chemistry on the Applied Biosystems (ABI) 433A peptide synthesizer according to the protocol used for the production of commercial PNA oligomers. The PNA oligomer GT-1a was synthesized at a 10 μmol scale using 106 mg of preloaded t-Boc-Lysine(Cbz) MBHA resin (˜0.1 μmol/mg). Synthesis of GT-1 was completed using ˜20 mg of the resin on which PNA GT-1a was synthesized. MS analysis of a test cleavage of the full length PNA oligomer showed a successful synthesis with little to no failure sequences present (observed m/z=4246.53, calculated m/z=4248.22).

Using the 433A, t-boc-aeg(Fmoc)-OH was coupled to ˜50 mg of the resin containing GT-1a PNA oligomer. The t-boc-aeg(Fmoc)-OH monomer was dissolved in N-methylpyrrolidinone (NMP) at 0.15M and double-coupled. The N-terminal Boc group was left intact, while the Fmoc protecting the secondary amine was removed with 20% piperidine/N,N′-dimethylformamide (DMF). Either Fmoc-glycine or Fmoc-β-alanine was double-coupled to the secondary amine, followed by Fmoc deprotection of the primary amine of the glycine or alanine moiety.

Guanylation of the Glycinyl (or β-Alaninyl) primary amine was performed using N,N′-bis-Cbz-guanylpyrazole (378.38 g/mol, Fluka P/N 56605). An approximate 30-fold excess of the guanylating reagent (22.7 mg or 60 μmol) was dissolved in 200 μL DMF:ACN, to which 10-20 μL of DIEA was added. The mixture was added to a pre-swelled PNA-resin (100 μL DMF) and reacted overnight while shaking. Both a Kaiser test and MS of a test cleavage confirmed guanylation of the glycine and β-alanine residues (truncated GT-2-Gly; observed m/z=3411.15, calculated m/z=3413.44. truncated GT-2-β-Ala; observed m/z=3426.57, calculated m/z 3427.47).

The remainder of each of the GT-2 sequences (i.e. one with a second guanidinylated glycine side chain and one with a second guanidinylated β-alanine side chain) was synthesized on the 433A as follows:

1) Initial removal of the t-boc from the N-terminus of the PNA oligomer was performed;

2) The t-boc-Thymine PNA monomer was coupled to the amine terminus of the PNA oligomer;

3) The t-boc-aeg(Fmoc)-OH monomer was coupled to the amine terminus of the PNA oligomer;

4) The Fmoc group of the secondary amine of the t-boc-aeg(Fmoc)-OH monomer was deprotected using piperidine in DMF as discussed above;

5) Either Fmoc-glycine or Fmoc-β-alanine was coupled to the secondary amine as discussed above (provided that in this case the Fmoc group of the Fmoc-glycine or Fmoc-β-alanine moiety was not removed);

6) The N-terminal t-Boc group of the PNA oligomer was deprotected;

7) t-boc-Thymine PNA monomer was coupled to the PNA oligomer and the N-terminal t-boc was left intact;

8) The Fmoc group of the previously coupled Fmoc-glycine or Fmoc-β-alanine was then deprotected with piperidine in DMF, as discussed above;

9) The resin was washed with dichloromethane (DCM).

The GT-2-Gly/1-Ala PNA-resins were then guanylated overnight by treatment with N,N′-bis-Cbz-guanylpyrazole and N,N′-diisopropylethylamine (DIEA) as described above. The PNA was cleaved from the support, precipitated and reconstituted as described in Example 1 above.

The crude PNAs were analyzed by both MS and HPLC. MS analysis was performed as described in Example 1, above. Analytical HPLC was performed using a 4×23 mm YMC ODS-AQ (C18) column with the following conditions: 0-20% B/20 min gradient at 1.5 mL min (0.1% TFA as modifier, A=H2O, B=ACN). The results are summarized in the table below.

| | MW | MW | HPLC | nmol |
|---|---|---|---|---|
| ID | Calculated | Observed | Purity | recovered |
| GT-2-Gly | 4144.15 | 4140.88 | 67.8% | 59.0 |
| GT-2-B-Ala | 4172.21 | 4165.63 | 75.0% | 68.3 |

Note: Values for the observed MW of the PNA oligomers were measured without performing a two point calibration.

### REFERENCES

1. Egholm et al., Nature 365: 566-568 (1993)
2. Kuhn et al., Nucl. Acids Res. 26: 582-587 (1998)
3. Corey et al., Biochemistry 41: 11118-11125 (2002)
4. Ly et al., J. Am. Chem. Soc., 125: 6878-6879 (2003)
5. Ly et al., Chem. Comm., 2: 244-246 (2005)
6. Nielsen et al., Tett. Lett. 39: 4707-4710 (1998)
7. Kiso et al., J.C.S. Chem. Comm. 770: 1063-1064 (1980)
8. Izdebski et al., J. Pep. Sci. 11: 60-64 (2005)
9. Chemomordik et al., J. Biol. Chem. 278: 585-590 (2003)
10. Thomas et al., J. Am. Chem. Soc. 86: 4452-4459 (1964)
11. Bernatowicz et al., Tett. Lett. 34: 3389-3392 (1993)
12. Rothbard et al., Proc. Natl. Acad. Sci. USA 97: 13003-13008 (2000)
13. Yong et al., J. Org. Chem. 62: 1540-1542 (1997)

While the present teachings are described in conjunction with various embodiments, it is not intended that the present teachings be limited to such embodiments. On the contrary, the present teachings encompass various alternatives, modifications and equivalents, as will be appreciated by those of skill in the art.

1. A method comprising:
a) reacting one or more primary or secondary amine groups of a PNA oligomer comprising a backbone with a reagent of formula I under basic conditions: 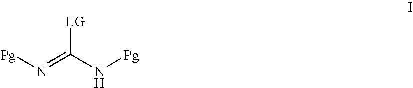
to thereby form a protected guanidinium group from each of said one or more reacted amine groups;
wherein,
each Pg is the same or a different amine protecting group;
LG is a leaving group that can be displaced by reaction with the one or more amine groups of the PNA oligomer; and
wherein each protected guanidinium group has the formula I′: 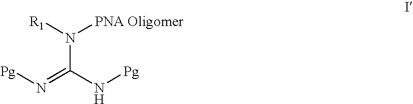
wherein, R₁ is hydrogen or an alkyl, alkenyl, alkynyl, heteroalkyl, heteroalkenyl, heteroalkynyl, heterocycloalkyl, aryl, heteroaryl, arylalkyl or heteroarylalkyl group.

2. The method of claim 1, further comprising deprotecting the one or more amine groups prior to its/their reaction with the reagent of formula I.

3. The method of claim 1, further comprising deprotecting one or both of the amine protecting groups, Pg, of the guanylated PNA oligomer.

4. The method of claim 3, wherein the deprotection of one or both of the amine protecting groups, Pg, is performed simultaneously with cleavage of the PNA oligomer from a solid support.

5. The method of claim 1, wherein LG is a group of formula II: 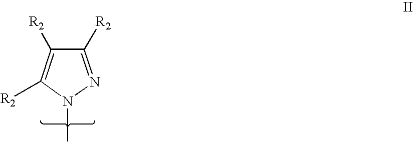
wherein the bracket “}” is used to indicate the points of attachment of the leaving group of formula II to the remainder of the reagent of formula I and wherein each R₂ is independently, hydrogen, fluorine, chlorine, bromine or iodine or an alkyl, an alkenyl, an alkynyl, a heteroalkyl, a heteroalkenyl, a heteroalkynyl, a heterocycloalkyl, an aryl, a heteroaryl, an arylalkyl, a heteroarylalkyl, a nitro (—NO₂), a cyano (—CN) or a sulfo (—SO₂) group.

6. The method of claim 1, wherein the reagent of formula I is generated in situ by combining reagents of formulas III and IV; 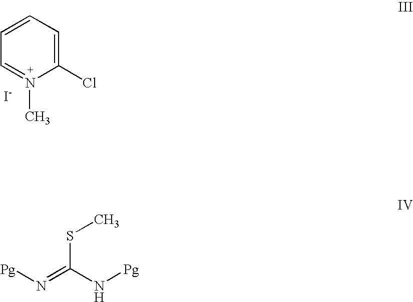
wherein each Pg is the same or a different amine protecting group.

7. The method of claim 1, wherein each Pg is independently, Aloc, Bhoc, Cbz, Cyoc, DDe, Fmoc or t-boc.

8. The method of claim 1, wherein the reagent of formula I is N,N′-bis-(t-boc)-guanylpyrazole (V) or N,N′-bis-Cbz-guanylpyrazole (VI)

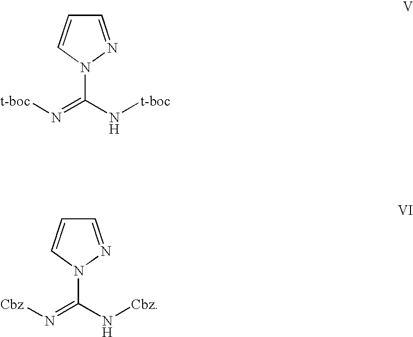

9. The method of claim 1, wherein the one or more amine groups is each independently a terminal amine group or a side chain amine group.

10. The method of claim 1, wherein at least one of the amine groups is linked to a side chain of a subunit of the backbone of the PNA oligomer wherein the backbone subunit has the formula VII: 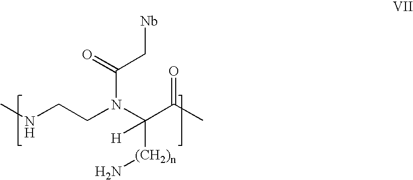
wherein n is an integer from 1 to 6 and Nb is a nucleobase.

11. The method of claim 10, wherein the at least one amine group is linked to the side chain of a subunit of the backbone of the PNA oligomer wherein the backbone subunit has the formula VIII: 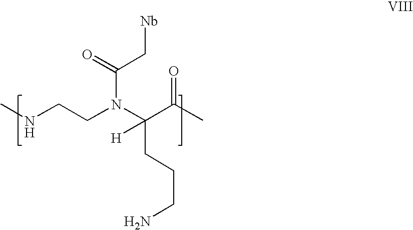
wherein Nb is a nucleobase.

12. The method of claim 11, wherein deprotection of both protecting groups Pg of the protected guanidinium group, after reaction of the PNA oligomer with the reagent of formula I according to claim 1, produces a backbone subunit of formula VIII′ or a protonated version thereof represented by formula VIII″: 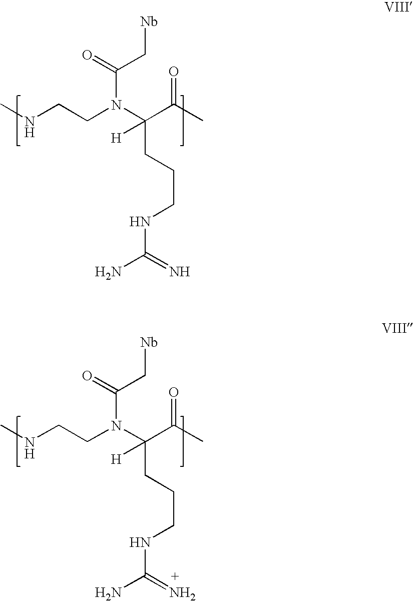
wherein, Nb is a nucleobase.

13. The method of claim 1, wherein at least one amine group is linked to a side chain of a subunit of the backbone of the PNA oligomer wherein the backbone subunit has the formula IX: 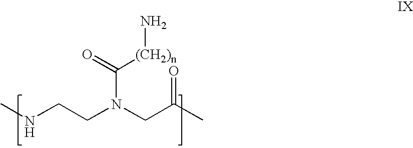
wherein n is an integer from 1 to 6.

14. (canceled)

15. The method of claim 1, wherein at least one of the amine groups is an N-terminal amine group of the PNA oligomer.

16. (canceled)

17. The method of claim 1, wherein the PNA oligomer is reacted with the reagent of formula I when it is support bound.

18. The method of claim 1, wherein the PNA oligomer is reacted with the reagent of formula I when it is in solution.

19. The method of claim 1, wherein the PNA oligomer comprises at least one covalently linked fluorophore and/or at least one covalently linked quencher.

20. The method of claim 1, wherein the PNA oligomer comprises alternating subunits wherein every other PNA subunit comprises an amine group linked to a side chain of the PNA backbone wherein each said side chain amine group is capable of being guanylated by reaction with the reagent of formula I under basic conditions.

21. The method of claim 20, wherein the PNA oligomer comprises four PNA subunits of formula XI′: 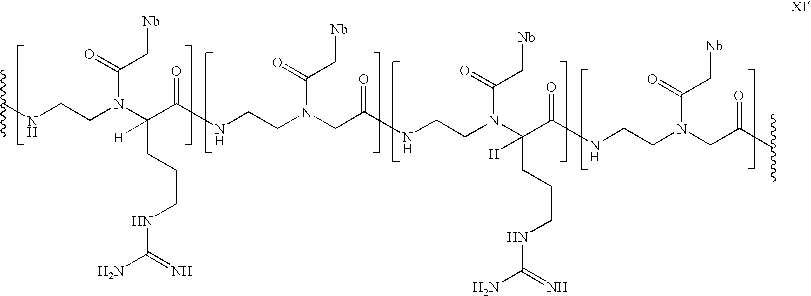
or the protonated form thereof represented by formula XI″: 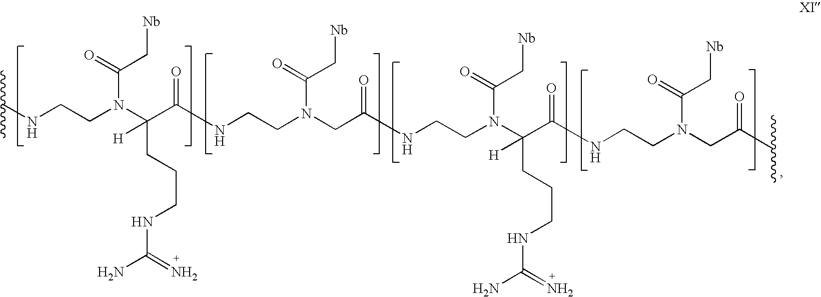
wherein the structure within each [ ] represents a PNA backbone subunit, each [symbol] indicates a point of attachment to the remainder of the PNA oligomer and each Nb is a nucleobase, wherein each nucleobase can be the same of different as compared with the other nucleobases of the PNA oligomer.

22. A PNA oligomer comprising at least one guanidinium group and at least one covalently linked fluorophore and/or covalently linked quencher.

23. The PNA oligomer of claim 22, wherein the PNA oligomer comprises at least one PNA subunit of formula VIII′: 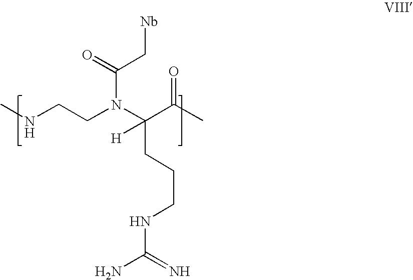
or a protonated version thereof represented by formula VIII″:

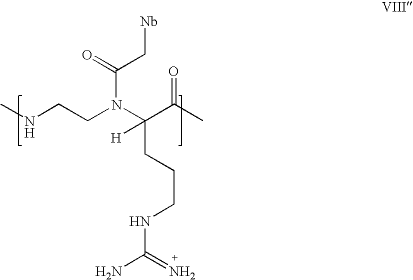

24. The PNA oligomer of claim 22, comprising at least one covalently linked dabcyl quencher and at least one PNA subunit of formula VIII′: 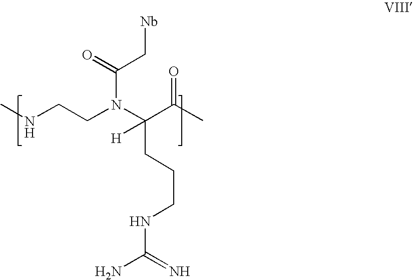
or a protonated version thereof represented by formula VIII″: 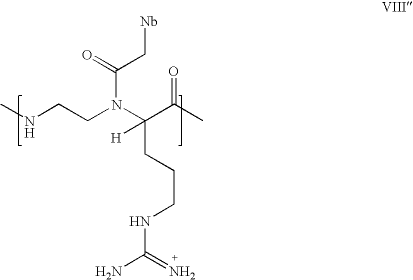
wherein Nb is a nucleobase.

25. (canceled)

26. A method comprising:
a) performing an amplification reaction using a PNA oligomer probe capable of sequence specifically hybridizing to a target sequence in a nucleic acid of interest, wherein said nucleic acid of interest can be amplified, if present, and wherein the PNA oligomer probe has the general formula XV, or the protonated form thereof represented by formula XV′: 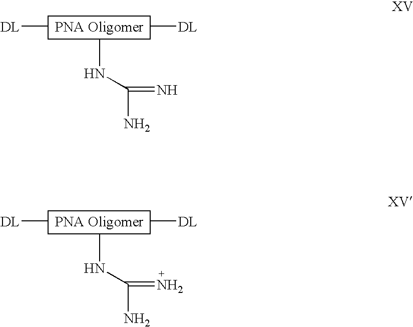
wherein each DL is independently of the other, a covalently linked fluorophore or a covalently linked quencher provided that only one of the two DLs can be a quencher.

27. The method of claim 26, wherein the PNA oligomer comprises at least one PNA subunit of formula VIII′: 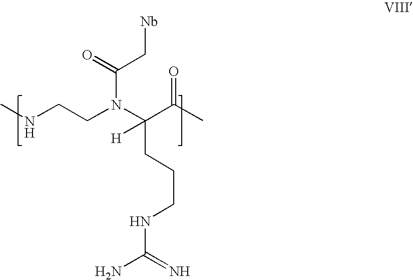
or a protonated version thereof represented by formula VIII″: 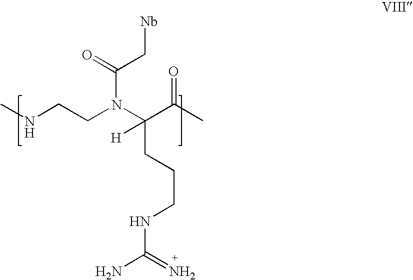
wherein Nb is a nucleobase.

28. (canceled)

29. The method of claim 26 wherein the amplification reaction is a Polymerase Chain Reaction (PCR), Ligase Chain Reaction (LCR), Strand Displacement Amplification (SDA), Transcription-Mediated Amplification (TMA), Q-beta replicase amplification (Q-beta) or Rolling Circle Amplification (RCA)

30-32. (canceled)
